(19) United States
(12) Patent Application Publication
(10) Pub. No.: US 2015/0305854 A1
(43) Pub. Date: Oct. 29, 2015

(54) VARIABLE SURFACE BREAST IMPLANT

(71) Applicant: Allergan, Inc., Irvine, CA (US)

(72) Inventors: David Schuessler, Santa Ana, CA (US); Dennis Van Epps, Goleta, CA (US)

(21) Appl. No.: 14/795,106

(22) Filed: Jul. 9, 2015

Related U.S. Application Data
(63) Continuation-in-part of application No. 14/106,524, filed on Dec. 13, 2013.
(60) Provisional application No. 61/736,687, filed on Dec. 13, 2012.

(51) Int. Cl. A61F 2/12

(52) U.S. Cl. CPC A61F 2/12; A61F 2210/0061; A61F 2210/0076; A61F 2230/0071; A61F 2250/0003; A61F 2250/0025

(57) ABSTRACT

A variable textured breast implant is provided including a front surface that has a porous texture and a back surface having a smooth or less porous texture than the front surface texture.

This application is a continuation-in-part of U.S. patent application Ser. No. 14/106,524, filed on Dec. 13, 2013, which claims priority and the benefit of U.S. Provisional Patent Application No. 61/736,687, filed Dec. 13, 2012, the entire disclosure of each of these applications being incorporated herein by this reference.

The present invention generally relates to breast implant technology and more specifically relates to a variable surface breast implant.

### BACKGROUND

Soft tissue implants are commonly used for a wide variety of clinical and cosmetic purposes. One use involves reconstructive applications that rebuild and restore a body part or structure to correct deformities from congenital anomalies, trauma, cancer, infections, disease, or medication side effects. The soft tissue implant serves to replace lost tissue, provide aesthetic improvement, support surrounding tissue and/or to maintain the normal appearance of the body. The restoration of a normal appearance has a significant beneficial psychological effect on post-operative patients, alleviating much of the shock and depression that often follows extensive surgical procedures. Another use involves augmentation applications that alter a body part or structure usually to improve its cosmetic or aesthetic appearance. Augmentation of the appearance also has beneficial psychological effects that improve self-esteem, well-being, and confidence of an individual.

The use of implantable prostheses for breast shaping, for example, for breast reconstruction following traumatic or surgical loss of breast tissue or, electively to increase volume of the breast is well known. Typically, the prosthesis or implant comprises of a soft, flexible envelope containing a liquid or gelatinous material. The envelope is commonly made from silicone or other bio-compatible polymer with varying degrees of elastic memory and permeability. These prostheses are filled with saline, and silicone oil or gel to mimic the tone and feel of natural breast tissue.

Capsular contracture is an adverse event related to breast implant surgery. It is believed to be a result of the immune system response to the presence of a foreign material in the body. A normal response of the body to the presence of a newly implanted object, for example a breast implant, is to form periprosthetic tissue, sometimes in the form of a capsule containing collagen fibers around the implant. Capsular contracture occurs when the capsule begins to contract and constrict the implant. This contracture can be discomforting or even extremely painful, and can cause distortion of the appearance of the augmented or reconstructed breast.

The exact cause of contracture is not known. However, some factors that may influence contracture include bacterial contamination of the implant prior to placement, submuscular versus subglandular placement, and smooth surface implants versus textured surface implants, the type or degree of texture on the implant surface, and bleeding or trauma to the area.

Surface texturing has been shown to reduce capsular contracture when implants are placed in the subglandular position compared to what are known as “smooth” surface implants. In other words, it is generally well known in the art that patients fitted with textured implants are less likely to exhibit contracture, relative to patients fitted with non-textured or smooth surface implants placed subglandularly. However, there is still a need for a textured implant that is specifically designed to encourage optimal tissue integration in the most beneficial location, and potentially reduce capsule formation and collagen fiber alignment described herein.

Conventional manufacturing processes for textured implants include the application of dissolvable particles onto a tacky elastomeric surface of an implant shell and subsequent removal thereof, leaving a dimpled or textured surface in the elastomer. Alternating layers of particles and elastomer provide a way to produce a textured surface defined by a porous surface having a desired depth.

There remains a need for better breast implants with textured regions.

### SUMMARY

Accordingly, devices and methods for making textured breast implants are provided. The devices and methods are more specifically directed to implants with variable surfaces, including textured surfaces. Variable textured breast implants are also provided.

The present invention provides methods for making prostheses, or soft tissue implants, for example, implants suitable for use in reconstruction or augmentation of the human breast. Breast implants made in accordance with the methods and devices described herein, generally comprise a soft elastomeric exterior and a gel or saline core or filling. The exterior has a variable texture for enhancing adhesion of tissue in desired regions of the implant.

In one aspect, methods are provided for texturing a breast implant exterior surface. The exterior of a breast implant can be generally defined by a posterior surface which generally faces the chest wall, and an anterior surface which faces away from the chest wall and when the implant is implanted in a breast. Conventionally, both the anterior surface and the posterior surface are identical to each other in terms of surface texture or lack thereof. In accordance with one aspect of the invention, the devices and methods are useful for making a breast implant having a posterior surface and an anterior surface that are different from one another, for example, have different textures from one another, or have texture on one surface and relative lack of texture on another surface.

In another aspect, a device for use in molding a variable textured breast implant is provided. The device generally comprises a member structured to engage a breast implant mandrel having a molding surface and a stem depending therefrom. The member includes a base-engaging distal portion and a stem-engaging proximal portion depending from the base-engaging distal portion. The base-engaging distal portion comprises a base-engaging surface, and an aperture defined in the base-engaging surface for receiving the stem of the mandrel when the mandrel is seated in the base engaging portion. The member is made of a material and is structured such that, when coupled with a breast implant mandrel, it provides a barrier for preventing silicone dispersion and/or texturing particles from contacting a portion of the mandrel when the mandrel is immersed in a silicone dispersion and/or texturing particles. In one embodiment, the member includes a proximally sloping rim circumscribing the base-engaging surface. The member may be made of any suitable material, for example, but not limited to an elastomeric material such as a silicone elastomer, or alternatively, a relatively rigid polymeric material, such as Acetal.

In one aspect the base-engaging surface of the member is configured or shaped to conform to a base region of breast implant mandrel. In one embodiment, the base-engaging surface is a substantially conically shaped surface. The aperture may be disposed generally in the center of the conically shaped surface.

In one embodiment, the proximally sloping rim slopes at an angle substantially equal to a slope of the mandrel surface, such that the proximally sloping rim substantially aligns with the mandrel surface when the mandrel is seated in the base-engaging portion.

In some embodiments, the proximally sloping surface has a slope angle relative to a plane perpendicular to the longitudinal axis of the member, the slope angle being between about 20° and about 70°, for example, between about 30° and about 60°. In one embodiment, the slope angle is about 45°.

In another aspect of the invention, a breast prosthesis shell is provided that includes a variable textured surface, for example, a shell made using the methods and devices described herein.

In yet another aspect of the invention, a method for making a textured breast implant is provided, the method comprising the steps of securing the device described elsewhere herein to a breast implant mandrel coated with a tacky elastomer, contacting the tacky elastomer with texturing particles, for example, by immersing the breast implant mandrel and device secured thereto into a bath of texturing particles; removing the device from the mandrel; curing the tacky elastomer; and removing the texturing particles to obtain a breast implant shell having a first textured surface and a second surface, for example a smooth surface, different from the first textured surface.

In one aspect of the invention, a breast implant is provided, the implant comprising an elastomeric envelope having a posterior surface defined by a cellular texture having a first depth defined by interconnected pores, the first depth being at least three pores in depth, and an anterior surface defined by an open cell texture having a second depth defined by pores, the second depth being about one pore in depth.

In yet another aspect of the invention, an assembly is provided for use in making a variable textured breast implant, the assembly comprising a mandrel having a molding surface and a stem depending therefrom and a device for receiving the mandrel as disclosed elsewhere herein.

In another aspect of the invention, a breast implant is provided having more significant texturing on the front, or anterior side, of the implant, and is reduced or omitted on the back, or posterior side of the implant. It has been discovered that this structure and arrangement of surface texturing provides a breast implant with certain surgical and medical advantages. In addition, the implant may provide a reduced risk of capsular contracture relative to conventional implants.

For example, when placed subglandularly, the implant structure will provide enhanced tissue adherence on front of the implant and to reduced tissue adherence to tissue, for example, muscle tissue, on the back of the implant.

Each and every feature described herein, and each and every combination of two or more of such features, is included within the scope of the present invention provided that the features included in such a combination are not mutually inconsistent.

### BRIEF DESCRIPTION OF THE DRAWINGS

These and other aspects, features and advantages of the present invention may be more clearly understood with reference to the following Detailed Description when considered in conjunction with the accompanying Drawings, of which:

FIG. 1 is a perspective view of an assembly for use in making a variable textured breast implant;

FIG. 2 is a view similar to FIG. 1, with a portion of the assembly shown in cross-section;

FIG. 3 is a perspective view of the assembly shown in FIG. 1, as being used to form a variable textured breast implant shell;

FIG. 4 is a partial cross-sectional side view of the assembly as being used to form a variable textured breast implant shell, as shown in FIG. 3; and

FIG. 5 is a side view of a breast implant formed with the assembly shown in FIG. 3;

FIG. 6 is a cross sectional view of another implant formed with the assemblies, devices or methods of the invention;

FIGS. 7, 7A and 7B illustrate a method of the invention for producing a variable textured breast implant;

FIGS. 8, 8A and 8B illustrate another method of the invention for producing a variable textured breast implant; and

FIGS. 9, 9A and 9B illustrate yet another method of the invention.

### DETAILED DESCRIPTION

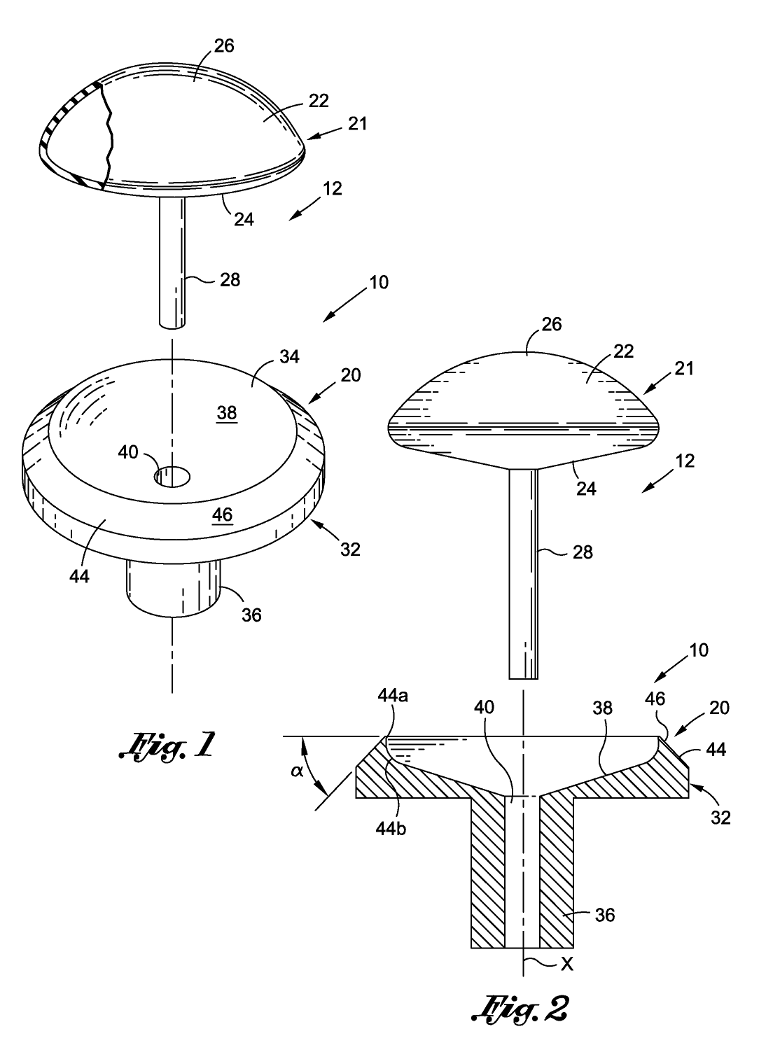

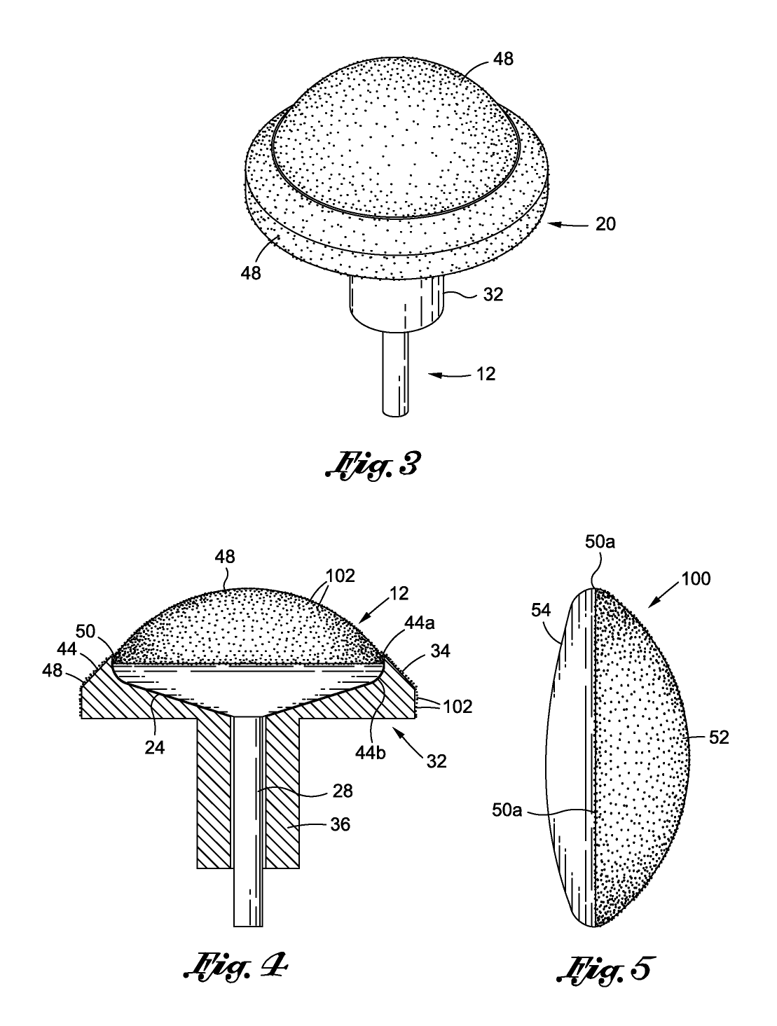

Turning now to FIGS. 1 and 2, an assembly of the invention is shown generally at 10, and comprises a mandrel 12, and a device 20 for use in molding a variable textured breast implant when used in conjunction with the mandrel 12.

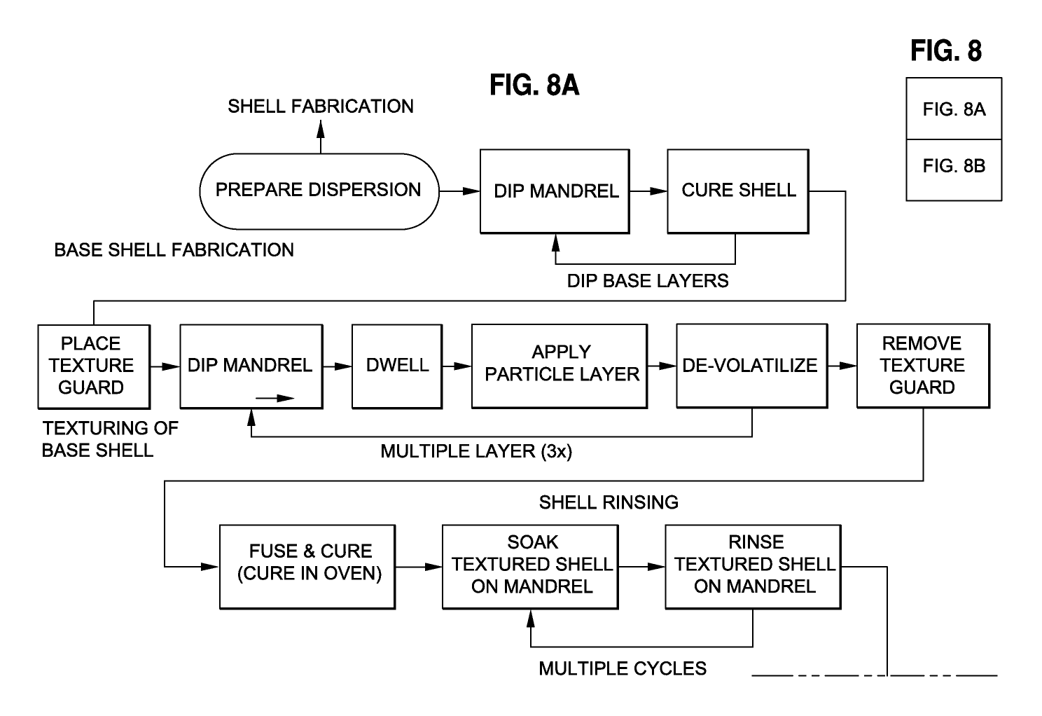

The mandrel 12 may be similar or identical to a standard, conventional breast implant mandrel 12, generally including a shaped form 21 defining a molding surface 22 having a proximal region 24 and a distal region 26 which together define a configuration of a breast implant shell 100 (shown, for example, in FIG. 6) to be molded therefrom. The mandrel further includes a stem 28 secured to the form 21 and depending in a proximal direction, for enabling manipulation of the mandrel 12 during the molding process.

In the shown embodiment, device 20 comprises a member 32 structured to engage a breast implant mandrel, such as mandrel 12. Member 32 generally includes a base-engaging distal portion 34 and a stem-engaging proximal portion 36 depending from the base-engaging distal portion 34. The base-engaging distal portion 34 comprises a base-engaging surface 38, an aperture 40 defined in the base-engaging surface 38, and a rim 44 circumscribing the base-engaging surface 38. The base engaging surface 38 has a shape in conformance with a portion of the shaped form 21, for example, is conically shaped as shown most clearly in FIGS. 1, 2 and 4.

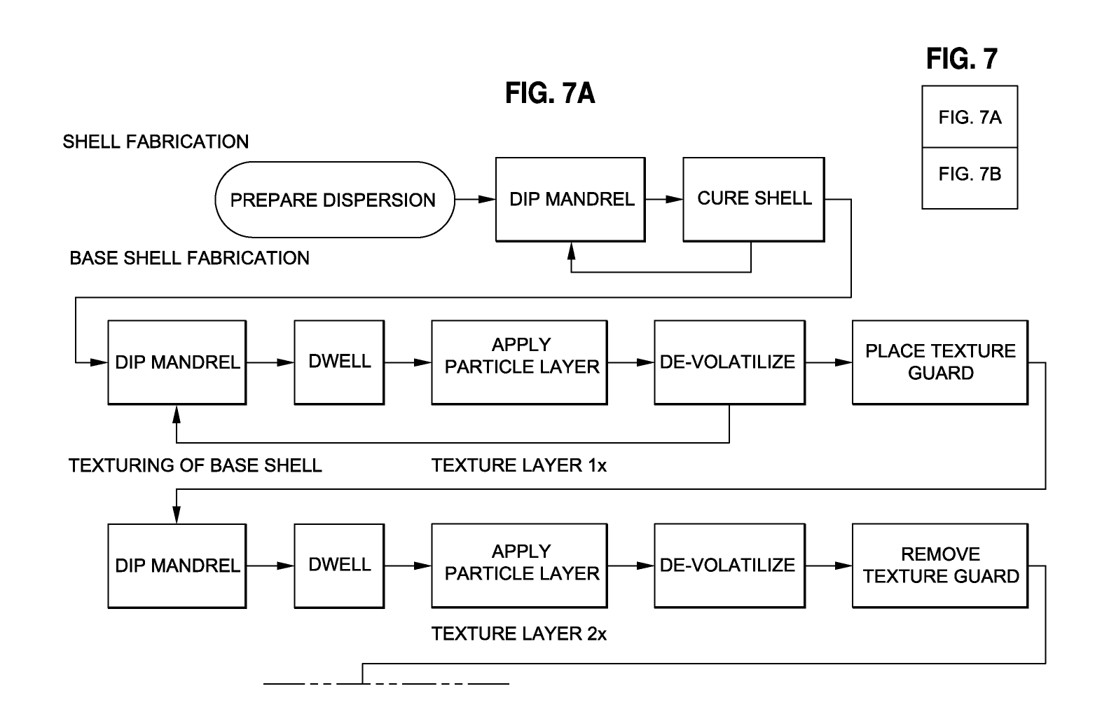

Turning briefly as well to FIG. 4, the stem-engaging proximal portion 36 of member 32 is structured to receive the stem 28 of mandrel 12 when mandrel 12 is seated in the base-engaging portion 34. As shown in FIG. 4, member 32 provides a shield or barrier for preventing silicone dispersion and/or texturing particles from contacting or adhering to a portion of the mandrel 12, for example, mandrel proximal region 24, when the mandrel 12 is immersed in a silicone dispersion and/or texturing particles 102.

Member 32 is configured and structured to provide a compliant, mating seal between a silicone elastomer covered mandrel 12 and base-engaging surface 38. Turning back now to FIG. 2, rim 44 comprises a surface 46 which slopes in a proximal direction as shown, having a slope angle (α) relative to a plane perpendicular to the longitudinal axis X of the member 32. The slope angle is between about 20° and about 70°, for example, between about 30° and about 60°. In one embodiment, the slope angle (α) is about 45°.

Turning specifically to FIG. 2, configuration of rim 44, having a sharp edge 44a and concave region 44b, may be configured to enhance sealing between member 32 and mandrel 12, for example, by providing a higher unit pressure against mandrel. The radius of curvature (R) of concave region 44b, in the shown embodiment, is about 0.34″.

Slope angle α may function in part to facilitate effective draining of particles and prevent pooling of texture particles 102 along rim 44 during the texturing process described hereinafter. For example, the proximally sloping rim 44 slopes at an angle substantially equal to a slope of the mandrel surface, such that the proximally sloping rim substantially aligns with the mandrel surface when the mandrel is seated in the base-engaging portion, as shown most clearly in FIG. 4.

In one aspect of the invention, methods are provided for making a variable textured breast implant shell, for example, using the assembly 10 shown in FIGS. 1 and 2.

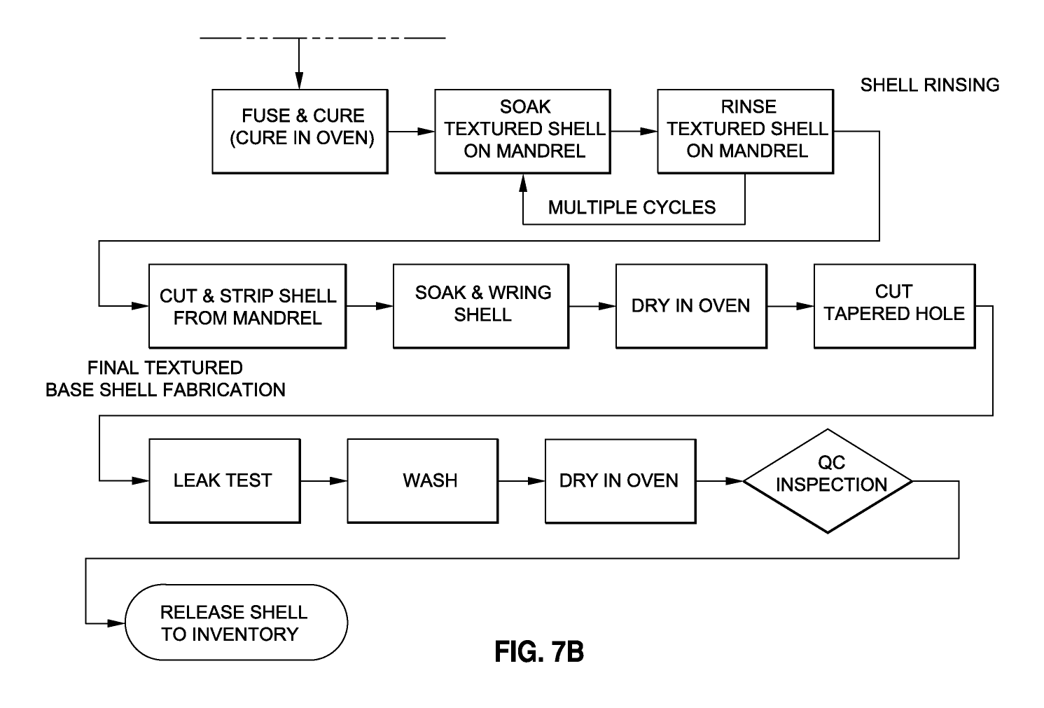

In one embodiment useful for making a breast implant 100 having a textured surface on one side thereof and a smooth or untextured surface on an opposing side thereof, such as shown in FIG. 5, the method may include providing a breast implant mandrel 12 having a cured silicone elastomer coating thereon, securing the device 20 to the mandrel 12, coating the breast implant mandrel with a tacky elastomer, such as a silicone dispersion, for example, by dipping, spraying or other suitable means, and immersing the breast implant mandrel 12 and device 32 secured thereto into a bath of texturing particles. Texturing particles may be any suitable texturing particles known in the art, for example, salt crystals, sugar crystals, starch beads, dissolvable polymeric beads, etc. Certain advantageous texturing particles useful with the present devices and methods are described in U.S. patent application Ser. No. 13/104,811 filed on May 21, 2012, and U.S. patent application Ser. No. 13/631,091, filed on Sep. 28, 2012, the entire content of each of these applications incorporated herein by this specific reference.

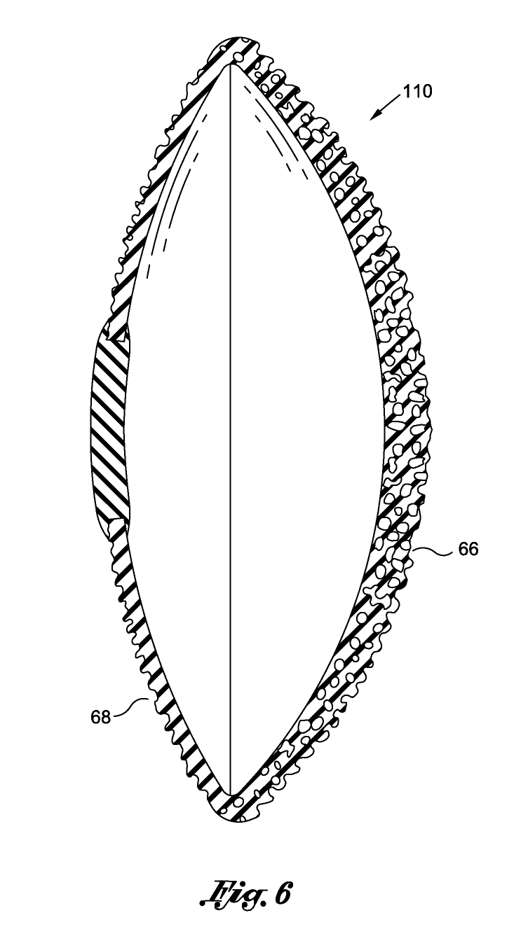

The assembly 10 at this point in the process is illustrated in FIGS. 3 and 4, which show mandrel 12 and member 32 having an elastomer particle-coated construction 48, as shown. The application of elastomer and particles can be repeated to achieve a desired thickness of texturing on the shell. The assembly 10 is then placed under curing conditions, for example, is placed into a curing chamber, for example, a heated oven, for a suitable amount of time, to cause at least partial curing of the elastomer coating. In this step, in some embodiments, the texturing particles may begin to melt and adhere to one another, which will provide an open cell, or interconnected cell texture when the particles are removed (see briefly, for example, FIG. 6).

Further, in some embodiments, the curing step is performed so that the elastomer is not fully cured at this point, but remains somewhat soft and/or tacky. This may be helpful to facilitate separation of the mandrel 12 from the member 32. Further still, optionally, a cutting implement may be used to sever the elastomer along a juncture 50 between the mandrel 12 and member 32, to produce a clean seam 50a between textured and untextured portions of the implant shell 100, for example, as shown in FIG. 5.

After the curing step, the texturing particles may then be removed from this construction 48 by any suitable means, for example, rinsing or dissolving in a solvent, leaving a textured porous surface. Member 32 is removed from the mandrel 12, for example, by sliding member 32 off of mandrel stem 28, and the elastomer/particle layered construction 48 is carefully stretched apart from and removed from the shaped form 21, resulting in a hollow, flexible envelope. In some embodiments, the particle removal step may be performed after the construction 48 is removed from the mandrel. Once the particles are removed from the elastomer, the construction 48 is useful as a breast implant shell 100 having one side, for example an anterior side 52 that is textured with cavities remaining from the removed particles, and an opposing side, for example, a posterior side 54, that is smooth and untextured, such as shown in FIG. 5. Finishing steps that are conventional may be performed to seal the aperture left from the mandrel stem with a patch, for example, and the shell 100 may be filled with saline or gel in any conventional manner.

The present invention can be modified to achieve a number of different types of breast implant shells having different forms of variable texture. For example, in some embodiments, a breast implant shell 110, shown in FIG. 6, having a first textured surface 66 produced by multiple layers, for example, three or more layers of particles/elastomer, and a second textured surface 68 produced by relatively fewer layers, for example, one layer, of particles/elastomer, may be provided. This may be accomplished substantially as described elsewhere herein, with one difference being that member 32 may be installed on mandrel 12 when the mandrel already includes a particle/elastomer covered surface, for example, a single layer of particles on the entire implant surface including the portion covered by the member 32. After installing of the member 32, further layers of particles and elastomer may be layered onto the mandrel/member assembly, while the member 32 is in place providing a shield from elastomer and particle application on exposed portions.

The resulting shell 110, after removal of particles, is shown in cross-section in FIG. 6. In this embodiment, the first textured surface 66 defines the anterior surface of the shell, outwardly facing breast tissue when implanted, and the second textured surface 68 defines the posterior surface of the shell, rearwardly facing the chest wall.

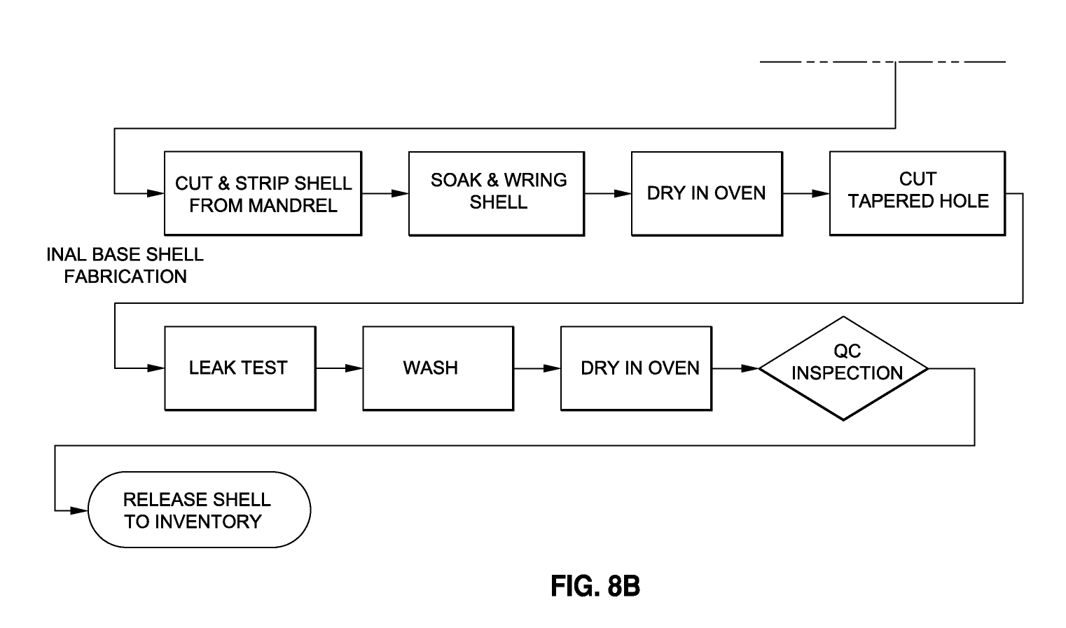

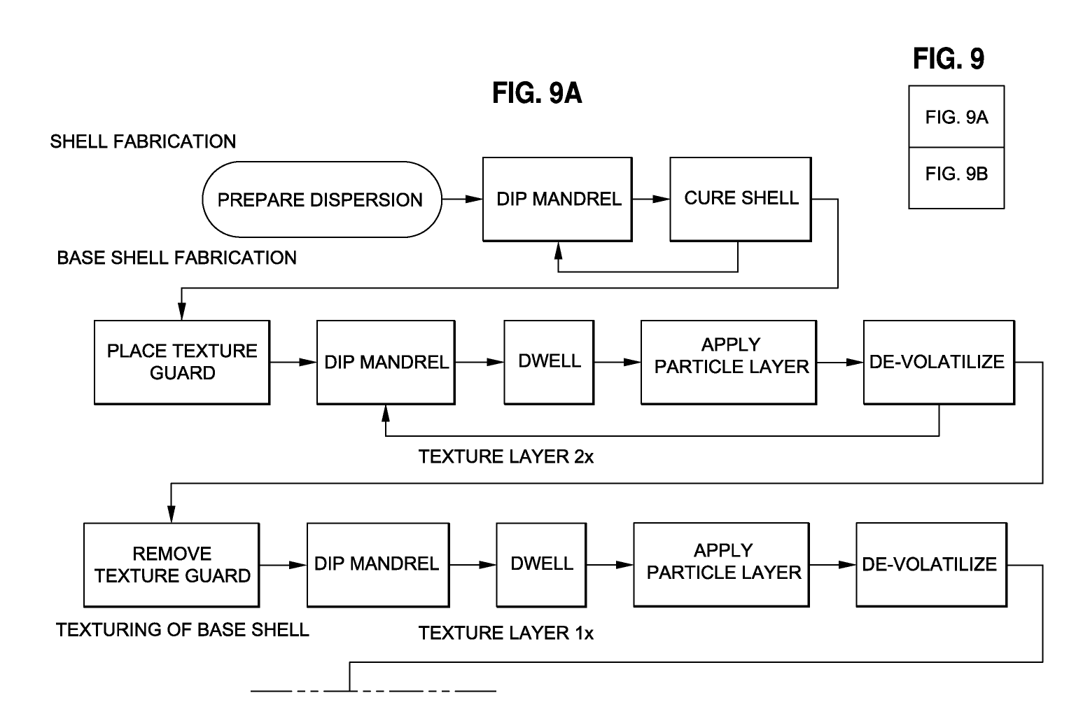

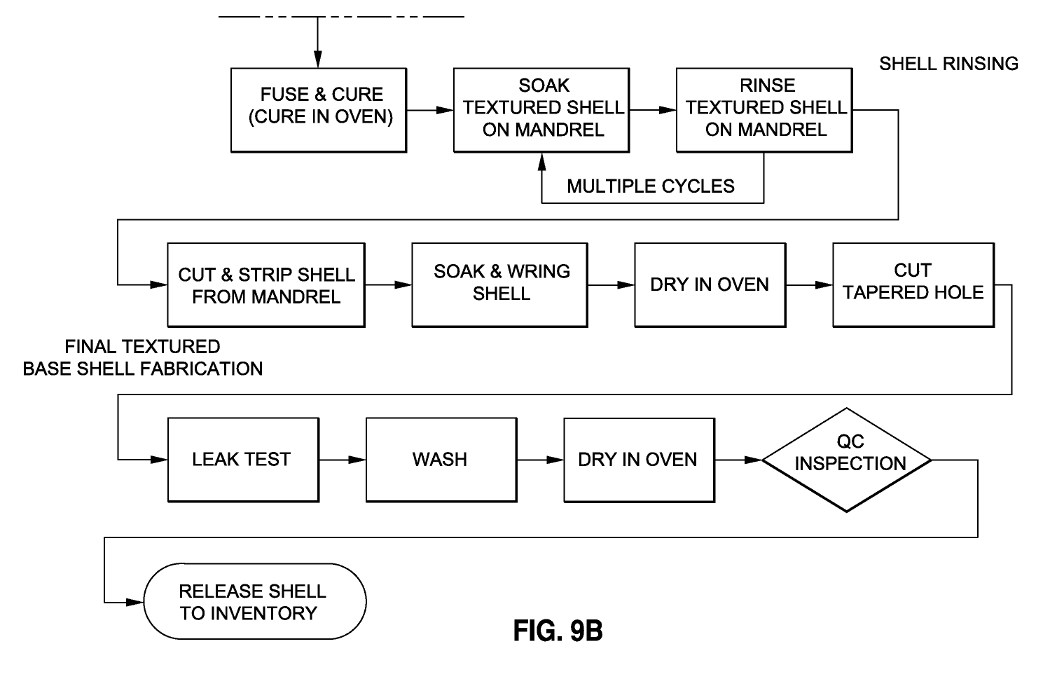

Other exemplary methods of the invention are shown as Flow Diagrams in FIGS. 7-9. The method shown in FIG. 7 (FIGS. 7A and 7B) can be used to create a breast implant shell having a single layered texture on one side of the implant, e.g. the posterior surface, and a double layered texture on another side of the implant, e.g. the anterior surface. The method shown in FIG. 8 (FIGS. 8A and 8B) can be used to create a breast implant shell, for example, shell 100, having a smooth surface on one side of the implant, e.g. the posterior surface, and a triple layer of texture on another surface, e.g. the anterior surface. The method shown in FIG. 9 (FIGS. 9A and 9B) can be used to create an implant shell having as single layer of texture on one surface and a double, triple, or greater number of layers of texture on an opposing surface, for example, such as implant 110 shown in FIG. 6.

In yet another aspect of the invention, a breast implant is provided having a variable textured surface that provides certain advantages over conventional breast implants, for example, fully textured implants. The implant may be manufactured using the assemblies, devices and methods disclosed herein, or other methods known in the art.

The implant generally comprises an anterior surface having a multilayered texture defined by open cell pores of a first depth, and a posterior surface having a single layered texture defined by open cell pores of a second depth that is less than the first depth.

For example, in some embodiments, the anterior surface of the implant includes a deeper texturing, for example a depth of porosity that is greater than texturing on the rear or posterior surface of the implant. Implant 110, shown in FIG. 6, provides an example of this embodiment of the invention. For example the anterior surface 66 of the implant comprises two, three, four, five or more layers of texturing, made for example, by alternating layers of texturing particles with silicone dispersion layers, during the manufacturing process; and the posterior surface 68 includes one layer of texturing made by application of a single layer of texturing particles (e.g. not alternating multiple layers of particles and elastomer during manufacturing).

Alternatively, the anterior surface includes two or more layers of texturing and the posterior surface has less or no texturing (smooth).

In any of the aforementioned embodiments, the depth of the porous structure of first surface region may be a relatively deep porous structure, for example, a porous structure that is multiple pores deep, for example a depth of porosity that is about 3, 4 or more pores in depth, such as shown in FIG. 6.

For example, the anterior region of the shell may be defined by a first textured surface defined by multiple layers of interconnected pores, for example, about two to about five layers of interconnected pores. The layers of interconnected pores may extend a first depth, for example, a depth of between about 0.2 mm to about 5.0 mm into the shell outer surface. In contrast, the posterior region of the implant may be defined by smooth, non-textured surface, or a less textured surface, for example, a substantially non-porous surface, for example, a dimpled surface.

In some embodiments, the anterior surface is defined by multiple layers of pores, while the posterior surface is defined by multiple layers of pores that extend less deeply into the shell than the anterior surface pores. In other words, in this embodiment, both the anterior and posterior of the implant are defined by a porous, textured structure, but the anterior surface is defined by a less deeply textured porous structure than the posterior surface.

In some embodiments, the pore size, for example, the pore diameter, is based on the size of the particles used to form the texture. In some embodiments, about 50%, or about 70% or about 80% or about 90% of the particles used to form the texture are generally spherical beads, for example, dissolvable polymer, sugar, salt or other dissolvable material beads, having a diameter of between about 100μ to about 1000μ, for example, about 200μ to about 800μ, for example, or about 300μ to about 700μ, or about 400μ to about 600μ in diameter.

In one embodiment, about 90% of the particles used to form the pores are between about 420μ to about 595μ in diameter.

In one embodiment, a variable textured breast implant is provided, the implant comprising an elastomeric shell comprising an anterior region and a posterior region, wherein the anterior region and posterior region define an outer surface of the shell, the anterior region defined by a first textured surface conducive to tissue ingrowth and further defined by interconnected pores wherein the interconnected pores of the first textured surface extend at least about two to about five pore diameters deep into the shell outer surface, and the posterior region defined by a surface less conducive to tissue ingrowth than the first textured surface.

In some embodiments, the first textured surface comprises multiple layers of said interconnected pores extending to a depth of between about 0.2 mm to about 5.0 mm into the shell outer surface. In another embodiment, the first textured surface extends a depth of between about 0.8 mm to about 3.0 mm. In some embodiments, the pores of the first textured region are relatively uniform in size. In some embodiments, the pores of the first textured region are relatively uniform in size and have a diameter of between about 100μ to about 1000μ, or between about 200μ to about 800μ, or between about 300μ to about 700μ, or between about 400μ to about 600μ. For example, at least about 70% of the pores, or at least about 90% of the pores of the first textured region have a uniform diameter in one of these ranges. For example, in a specific embodiment, at least about 90% of the pores of the first textured region have a diameter of between about 400μ to about 600μ.

In accordance with one aspect of the invention, the surface of the posterior region is a smooth, non-textured surface. In another embodiment, the surface of the posterior region is defined by a dimpled surface. In some embodiments, the surface of the posterior region is defined by pores extending no greater than about one pore deep into the shell outer surface.

In these embodiments, the present implant 110 may provide certain surgical advantages over prior art implants. For example, when it becomes desirable or necessary to remove the implant from a patient, the implant can be removed with less trauma to the patient relative to conventional implants. For example, during surgical removal of the implant from a patient, the anterior portion of the implant can be readily accessed through the original incision, for example an inframammary incision. The integrated tissue on the posterior surface of the implant can be visualized and surgically separated from the more heavily or deeply textured surfaces of the implant. The posterior side of the implant, which is by nature more difficult to surgically access, can be separated from the tissue by simple finger dissection or peeling away of the implant due to its minimal tissue integration. Aesthetically, tissue adherence advantageously can maintain a desired position of the implant in the breast, preventing rotation or migration, and maintaining correct orientation of anatomically shaped implants.

Unless otherwise indicated or otherwise clearly contradicted by context, combinations of the above-described elements in all possible variations thereof are contemplated to be included within the scope of the invention.

What is claimed is:

1. A variable textured breast implant for placement in a breast, the implant comprising:
an elastomeric shell comprising an anterior region and a posterior region, wherein the anterior region and posterior region define an outer surface of the shell;
the anterior region defined by a first textured surface defined by interconnected pores wherein the interconnected pores of the first textured surface extend at least about two to about five pore diameters deep into the shell outer surface; and
the posterior region defined by a surface less conducive to tissue ingrowth than the first textured surface.

2. The implant of claim 1 wherein the first textured surface comprises multiple layers of said interconnected pores extending to a depth of between about 0.2 mm to about 5.0 mm into the shell outer surface.

3. The implant of claim 13 wherein the first textured surface extends a depth of between about 0.8 mm to about 3.0 mm.

4. The implant of claim 1 wherein the pores of the first textured region are relatively uniform in size.

5. The implant of claim 1 wherein the pores of the first textured region are relatively uniform in size and have a diameter of between about 100μ to about 1000μ.

6. The implant of claim 1 wherein at least about 70% of the pores of the first textured region have a diameter of between about 100μ to about 1000μ.

7. The implant of claim 1 wherein at least about 90% of the pores of the first textured region have a diameter of between about 100μ to about 1000μ.

8. The implant of claim 4 wherein the pores of the first textured region have a diameter of between about 200μ to about 800μ.

9. The implant of claim 4 wherein the pores of the first textured region have a diameter of between about 300μ to about 700μ.

10. The implant of claim 4 wherein the pores of the first textured region have a diameter of between about 400μ to about 600μ.

11. The implant of claim 1 wherein at least about 90% of the pores of the first textured region have a diameter of between about 400μ to about 600μ.

12. The implant of claim 1 wherein the surface of the posterior region is a smooth, non-textured surface.

13. The implant of claim 1 wherein the surface of the posterior region is defined by a dimpled surface.

14. The implant of claim 1 wherein the surface of the posterior region is defined by pores extending no greater than about one pore deep into the shell outer surface.

15. A variable textured breast implant for placement in a breast, the implant comprising:
an elastomeric shell comprising an anterior region and a posterior region, wherein the anterior region and posterior region define an outer surface of the shell;
the anterior region defined by a first textured surface defined by layers of interconnected pores extending a first depth into the shell outer surface; and
the posterior region defined by a second textured surface defined by pores extending to a second depth of less than said first depth.

16. The implant of claim 15 wherein the first depth is a depth of between about 0.2 mm to about 5.0 mm into the shell outer surface.

17. The implant of claim 15 wherein the pores of the first textured surface and the pores of the second textured surface are substantially the same in diameter.

18. The implant of claim 17 wherein the pores of both the first textured surface and the second textured surface generally have a diameter of between about 100μ to about 1000μ.

19. The implant of claim 17 wherein at least about 70% of the pores of both the first textured surface and the second textured surface have a diameter of between about 100μ to about 1000μ.

20. The implant of claim 17 wherein the pores of both the first textured surface and the second textured surface generally have a diameter of between about 400μ to about 600μ.

21. The implant of claim 17 wherein at least about 90% of the pores both the first textured surface and the second textured surface have a diameter of between about 400μ to about 600μ.

22. A variable textured breast implant for placement in a breast, the implant comprising:
an elastomeric shell comprising an anterior region and a posterior region, wherein the anterior region and posterior region define an outer surface of the shell;
the anterior region defined by a first textured surface conducive to tissue ingrowth and further defined by about two to about five layers of interconnected pores extending a first depth of between about 0.2 mm to about 5.0 mm into the shell outer surface, wherein at least about 90% of the pores have a diameter of between about 400μ to about 600μ; and
the posterior region defined by a smooth surface.
